SEQUENCE LISTING (10 sequences)

SEQ ID NO: 1 - RNA, 21 nt, Artificial sequence
  SiRNA oligonucleotide
aaguauuuucccauccccgug

SEQ ID NO: 2 - RNA, 21 nt, Artificial sequence
  SiRNA oligonucleotide
aaggaacuuuugaacuggcaa

SEQ ID NO: 3 - RNA, 21 nt, Artificial sequence
  SiRNA oligonucleotide
aauucccuguucagacuacau

SEQ ID NO: 4 - DNA, 780 bp, Homo sapiens
  misc_feature: Anti-sense sequence
atacgggatgatctggatgaccctggccttcttcatgtttgccggcaagttgcccgtcctcatgttctccttcatgatccggacatttgtcaatttcctgaactcctcctccagcccgatcttgtcgaagtgggtggtggtgccgtgcatggtctgcaggtgcttctccagggaggacacgtccagctctgtgtccccgtagaggtagtactcgagtaaggcttggtagatgaacgtgtactgcatatccgtttgaaccatctgagggcgctgattacggattcgagacacaaattcaaacacatccaccttctgctccgcgtgcatcatggccatcatggcatcgatcacaatgaaggtgcccgtccggcccacgcccgcgctacagtggaccacgatgggcccagcgtgcacggggttgagcgtctttactttcttgaggaacttcagcatcccaatgggggtaaaaggcactccgaagtcgggccagctggtgaagtgcagctgtgagaccagcctgggggctttgcagccgtcggggagctgtggctgtatgcagaacttccggatggtgtagtcgaccaaaaccacgcagtcctccacgcacacccggatgtttccataggtccagcagccttggtcgggccagtactgatggcacttttcctctttcctttctttcaagtttgttaacatgacgatggtcgcagacttttgctcccagaccattctccagaagtcgttaaccgtttcctgtttgggaccttgagctgctatgaa

SEQ ID NO: 5 - DNA, 23 bp, Artificial sequence
  pSUPER plasmid based siRNA
aagcaatcagcgtacgagacctt

SEQ ID NO: 6 - DNA, 23 bp, Artificial sequence
  pSUPER plasmid based siRNA
gattacacgatccgaaagttatg

SEQ ID NO: 7 - DNA, 23 bp, Artificial sequence
  pSUPER plasmid based siRNA
aagatcgggctggaggaagagtt

SEQ ID NO: 8 - DNA, 23 bp, Artificial sequence
  pSUPER plasmid based siRNA
aagcaggagacagtgaatgactt

SEQ ID NO: 9 - DNA, 23 bp, Artificial sequence
  pSUPER plasmid based siRNA
aaggaggagaagtgctacaagta

SEQ ID NO: 10 - DNA, 9 bp, Artificial sequence
  Plasmid based SiRNA linker
ttcaagaga

(19) United States
(12) Patent Application Publication
(10) Pub. No.: US 2009/0131349 A1
(43) Pub. Date: May 21, 2009

(54) Methods and Compositions for Modulating Body Weight and for Treating Weight Disorders and Related Diseases

(76) Inventors: Ari Elson, Rechovot (IL); Richard Klinghoffer, Seattle, WA (US)

Correspondence Address: Martin D. Moynihan;PRTSI, P.O. Box 16446, Arlington, VA, 22215, US

(21) Appl. No.: 11/885,633

(22) PCT Filed: Feb. 23, 2006
(86) PCT No.: PCT/IL06/00250

§ 371 Date: Jun. 6, 2008

Related U.S. Application Data
(60) Provisional application No. 60/657,696, filed on Mar. 3, 2005.

(51) Int. Cl. A61K 31/7088

(52) U.S. Cl. 514/44

(57) ABSTRACT

Methods of modulating body weight and/or fat content of a subject, methods of treating or preventing weight disorders and methods of treating or preventing weight disorder related diseases are disclosed. The methods include modifying an activity or expression of a protein tyrosine phosphatase epsilon (PTPe) so as to modulate the body weight and/or fat content of the subject. Pharmaceutical compositions and articles of manufacture useful in practice of the methods are further disclosed.

### FIELD AND BACKGROUND OF THE INVENTION

The present invention relates to methods, pharmaceutical compositions and articles of manufacture for modulating body weight and/or fat content and for treating or preventing weight disorders and related diseases in a subject.

Obesity, defined as an excess of body fat relative to lean body mass, represents the most prevalent of weight disorders affecting 30-50% of the middle aged population of the western world. Obesity is associated with important psychological and medical diseases including atherosclerosis, hypertension, Type II or non-insulin dependent diabetes mellitus, pancreatitis, hypercholestrosaemia, hypertriglyceridaema, and hyperlipidemia.

At present, only a limited number of treatment approaches are available for treating obesity. Exemplary treatment approaches and agents are disclosed in U.S. Pat. Nos. 3,867,539; 4,446,138; 4,588,724; 4,745,122; 5,019,594; 5,300,298; 5,403,851; 5,567,714; 5,573,774; 5,578,613; and 5,900,411. Although some prior art treatment approaches are effective in controlling obesity over a relatively short time period, unfortunately, such approaches have not been very successful in a long term treatment of this disorder.

Other weight disorders include anorexia nervosa and cachexia which are characterized by abnormal weight loss. Anorexia, which is characterized by a loss of appetite and loss of weight, affects approximately 0.2% of the female population of the western world, as well as the majority of male and female cancer patients. Cachexia, which is characterized by premature satiety, asthenia and loss of lean body mass, affects the majority of metastatic cancer patients and effectively contributes to their death. The weight loss associated with anorexia and cachexia is caused by a reduction in body fat stores as well as by a reduction in total body protein mass. Cachexia is also frequently associated with other chronic diseases such as cystic fibrosis and AIDS.

The prevention or treatment of underweight conditions remains a frustrating problem. Animal and human studies suggest that nutritional support is largely ineffective. For example, Brennan and Burt (Cancer Treatment Reports 65: 67-68, 1981) demonstrated marginal effects via parenteral nutrition treatment. Recently, U.S. Pat. No. 6,387,883 disclosed method for the prevention and treatment of cachexia and anorexia which include administering omega-3 fatty acids, branched-chain amino acids and an antioxidant.

There is thus a widely recognized need for, and it would be highly advantageous to have, novel and efficacious methods for treating weight disorders and related diseases.

While reducing the present invention to practice, the present inventors unexpectedly uncovered that modifying an activity or expression of protein tyrosine-phosphatase epsilon (PTPe) in test animals modifies their body weight and body fat content. Thus, the present invention provides novel methods, pharmaceutical compositions and articles of manufacture for modulating body weight and/or fat content of a subject and for treating or preventing weight disorders and related diseases.

### SUMMARY OF THE INVENTION

According to one aspect of the present invention there is provided a method of modulating a body weight and/or fat content of a subject, comprising modifying an activity or expression of the protein tyrosine phosphatase epsilon (PTPe) thereby modulating the body weight and/or fat content of the subject

According to another aspect of the present invention there is provided a method of treating or preventing a weight disorder in a subject, comprising modifying an activity or expression of PTPe, thereby treating or preventing the weight disorder in the subject.

According to yet another aspect of the present invention there is provided a method of treating or preventing a weight disorder related disease in a subject, comprising modifying an activity or expression of a PTPe thereby treating or preventing the weight disorder related disease in the subject.

According to still another aspect of the present invention there is provided a pharmaceutical composition for modulating a body weight and/or fat content of a subject, the pharmaceutical composition comprising, as an active ingredient, a therapeutically effective amount of an agent capable of modifying an activity or expression of a PTPe in the subject and a pharmaceutically acceptable carrier and/or excipient.

According to an additional aspect of the present invention there is provided an article of manufacture, comprising a packaging material and a pharmaceutical composition identified for use in modulating a body weight and/or fat content of a subject being contained within the packaging material, the pharmaceutical composition including, as an active ingredient, an agent capable of modifying an activity or an expression of a PTPe in the subject. and a pharmaceutically acceptable carrier.

According to yet an additional aspect of the present invention there is provided an article of manufacture, comprising packaging material and a pharmaceutical composition identified for use in treating or preventing a weight disorder of a subject being contained within the packaging material, the pharmaceutical composition including, as an active ingredient, an agent capable of modifying an activity or an expression of a PTPe in the subject. and a pharmaceutically acceptable carrier.

According to yet a further aspect of the present invention there is provided an article of manufacture, comprising packaging material and a pharmaceutical composition identified for use in treating or preventing a weight disorder related disease of a subject being contained within the packaging material, the pharmaceutical composition including, as an active ingredient, an agent capable of modifying an activity or an expression of a PTPe in the subject. and a pharmaceutically acceptable carrier.

According to still a further aspect of the present invention there is provided a method of identifying a drug candidate suitable for modulating a body weight or fat content of a subject, comprising screening a plurality of molecules for a molecule capable of modifying an activity or expression of a PTPe, the molecule capable of modifying an activity or expression being the drug candidate.

According to further features in preferred embodiments of the invention described below, the PTPe is a receptor or a non-receptor type PTPe.

According to still further features in the described preferred embodiments the PTPe is RPTPe, cyt-PTPe, p65 or p67.

According to still further features in the described preferred embodiments modifying an activity or expression of PTPe includes at least partially inhibiting an activity or expression of the PTPe.

According to still further features in the described preferred embodiments at least partially inhibiting an activity or expression of PTPe is effected by introducing into the subject an agent selected from the group consisting of: (i) a molecule which binds the PTPe; (ii) an enzyme which cleaves the PTPe; (iii) an antisense polynucleotide capable of specifically hybridizing with an mRNA transcript encoding the PTPe; (iv) a ribozyme which specifically cleaves transcripts encoding PTPe; (v) a small interfering RNA (siRNA) molecule which specifically cleaves PTPe transcripts; (vi) a non-functional analogue of at least a catalytic or binding portion of the PTPe; and (vii) a molecule which prevents PTPe activation or substrate binding.

According to still further features in the described preferred embodiments the small interfering RNA (siRNA) molecule includes SEQ ID NO. 7.

According to still further features in the described preferred embodiments modifying an activity or expression is accomplished via gene knockout.

According to still further features in the described preferred embodiments introducing is effected via systemic administration of the agent.

According to still further features in the described preferred embodiments the agent capable of modifying an activity or expression of a PTPe is a phosphatase inhibitor.

According to still further features in the described preferred embodiments the subject is a human.

According to still further features in the described preferred embodiments modifying an activity or expression of PTPe includes increasing the activity or expression of the PTPe.

According to still further features in the described preferred embodiments increasing the activity or expression of the PTPe is effected by an action selected from the group consisting of: (i) introducing into the subject an exogenous polynucleotide sequence designed and constructed to express at least a functional portion of the PTPe in the subject; (ii) increasing expression of endogenous PTPe in the subject; and (iii) increasing endogenous PTPe activity.

According to still further features in the described preferred embodiments the weight disorder is selected from the group consisting of obesity, anorexia and cachexia.

According to still further features in the described preferred embodiments the weight disorder related disease is selected from the group consisting of atherosclerosis, hypertension, Type II or non-insulin dependent diabetes mellitus, pancreatitis, hypercholestrosaemia, hypertriglyceridaema and hyperlipidemia.

According to still further features in the described preferred embodiments the screening is accomplished by measuring at least one parameter selected from the group consisting of PTPe binding, specific binding to a PTPe transcript, PTPe cleavage, and binding to a PTPe binding site.

According to still further features in the described preferred embodiments the screening is effected by at least one method selected from the group consisting of an antibody based assay, an assay for competitive inhibition of PTPe binding, an assay of inhibition of PTPe activity, an assay of specific PTPe binding, an assay of specific binding to at least a portion of an PTPe transcript, an assay of PTPe molecular weight and an assay of PTPe transcript amount.

The present invention successfully addresses the shortcomings of the presently known configurations by providing methods of modulating body weight and/or fat content and of treating or preventing weight disorders and related diseases in a subject. The methods include modifying an activity or expression of protein tyrosine phosphatase epsilon (PTPe) in the subject. The invention further relates to pharmaceutical compositions and articles of manufacture for clinical practice of the methods. In addition, the present invention provides methods of identifying drug candidates which may be suitable for use in methods, pharmaceutical compositions and articles of manufacture according to the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates the effect of PTPe and estrogen deficiencies on the body weight changes of mature female mice. PTPe knock-out (KO) and wild-type (WT) mice were given access to food and water ad libitum. Ovariectomy (OVX), or ovary manipulation (sham) surgery was performed on 10-11 month old mice and the body weight of each animal was recorded weekly for eight weeks following surgery. The line graph data points present means of 7-8 animals per genotype and treatment (replications). The body weight means of the WT-Sham mice were significantly higher (p<0.05) than the KO-Sham mice from week 0 throughout. The body weight means of the WT-OVX mice were significantly higher (p<0.05) than the WT-Sham mice from week 2 throughout. On the other hand, the mean body weight of the KO-OVX did not differ significantly from the KO-sham mice, indicating that the KO mice were essentially protected from any ovariectomy-induced body weight gain.

FIG. 2 illustrates the effect of PTPe and estrogen deficiencies on the body fat content of mature female mice. PTPe knock-out ((KO) and wild-type (WT) mice were given access to food and water ad libitum. Ovariectomy (OVX), or ovary manipulation (sham) surgery was performed on 10-11 month old mice and the body fat content (%) of each animal was measured pre-surgery and 8 weeks post-surgery. The bars present means of 7-8 animals per genotype and treatment (replications) and the standard deviations of the means.

FIG. 3 illustrates the effect of PTPe deficiency on the body weight of aged female mice. PTPe knock-out ((PTPE −/−) and wild-type (WT) mice were given access to food and water ad libitum during 14 months. The histogram (left) presents the final body weight means and standard deviations of 13 PTPe knock-out and 8 WT animal replications. The dot graph (right) presents the body weight values of individual animals. The average body weight of the PTPe knock-out mice was significantly (p<0.0001) lower than of the average body weight of the wild type mice.

FIG. 4 illustrates the effect of PTPe deficiency on the body fat content of aged female mice. PTPe knock-out (PTPE −/−) and wild-type (WT) mice were given access to food and water ad libitum over a 14 months period, then analyzed for the body fat content. The histogram (left) presents the percent body fat means of treatment groups and the standard deviation of the means. The histogram (left) presents the body weight means of 8 PTPe knock-out and 13 WT animal replications. The dot graph (right) presents the percent body fat content of individual animals. The percent body fat of the PTPe knock-out mice was significantly (p=0.0082) lower than the percent body fat of the wild-type mice.

FIG. 5 illustrates the effects of PTPe and estrogen deficiencies on the body weight of young female mice. PTPe knock-out ((KO) and wild-type (WT) mice were given access to food and water ad libitum. Ovariectomy (OVX), or ovary-manipulation (sham) operation, was performed on 2-3 months old mice and the body weight of each animal was measured over a 10 week period post operation. The graph data points present means of five animals per genotype and treatment (replications) and the bars present the standard errors of the means. The body weight means of the WT-OVX mice were significantly higher (p<0.05) than the WT-Sham mice in weeks 2-10 post operation. The body weight means of the WT-OVX mice were significantly higher (p<0.05) than the KO-OVX mice in weeks 4-10 post operation. On the other hand, the mean body weight of the KO-OVX mice did not differ significantly from the KO-sham mice, indicating that the KO mice were essentially protected from any ovariectomy-induced body weight gain.

FIG. 6 illustrates the effects of PTPe and estrogen deficiencies on the weekly food intake by young female mice. PTPe knock-out ((KO) and wild-type (WT) mice were given access to food and water ad libitum. Ovariectomy (OVX), or ovary manipulation (sham) operation, was performed on 2-3 month-old mice and the food consumption rate (grams per week) by each animal was measured over a 10 week period post operation. The graph data points present means of five animals per genotype and treatment (replications). The food intake means of the WT-Sham mice were significantly higher (p<0.05) than the KO-Sham mice in weeks 1 and 6-9 post operation. The food intake means of the WT-OVX mice were significantly higher p<0.05) than the KO-OVX mice in weeks 2 and 4-9 post operation. Overall, the rate of food intake by KO mice was significantly (p<0.05) lower than the food intake by the WT mice in weeks 4-10 post operation, regardless if the animals were ovariectomized or sham operated.

FIG. 7 illustrates the effect of PTPe deficiency on the body weight of male and female mice. PTPe knock-out (EKO) and wild-type (WT) mice were given access to food and water ad libitum and the body weight of each animal was measured over an 88-108 days period. The data points present means of 26-53 animals (replications) per sex and genotype and the bars present the standard errors of the means. The body weight means of the 42-120 days old WT male mice were significantly higher (p<0.001) than the EKO male mice of the same age. The body weight means of the 42-150 days old WT female mice were significantly higher (p<0.05) than the EKO female mice of the same age.

FIG. 8 presents the sequences of several siRNA molecules generated against PTPre.

FIG. 9 illustrates the results of screening of the siRNA molecules described in FIG. 8 for suppression of cyt-PTPe expression in 293 cells transfected with cyt-PTPe fused to GFP.

FIG. 10 illustrates suppression of cyt-PTPe expression by pSUPER siRNA in 293 cells transfected with cyt-PTPe.

FIG. 11 illustrates the specificity of siRNA-pSUPER PTPe inhibition. The siRNA inhibited expression of the two major protein isoforms of PTPe—the receptor-type and the non-receptor type forms (RPTPe and cyt-PTPe, respectively), in contrast, no inhibitory effect is observed when attempting to inhibit expression of the highly-related RPTP alpha.

### DESCRIPTION OF THE PREFERRED EMBODIMENTS

The present invention is of novel methods of modulating body weight and/or fat content and of treating or preventing weight disorders and related diseases in a subject. More particularly, the invention relates to modifying an activity or expression of protein tyrosine phosphatase epsilon (PTPe) thereby modulating body weight and fat metabolism in the subject. The invention further relates to pharmaceutical compositions and articles of manufacture for use in modulating body weight and/or fat content and for treating or preventing weight disorders and related diseases in a subject. In addition, the invention provides a method of identifying a drug candidate which can be used in modulating body weight and/or fat content of a subject. The principles and operation of the methods, pharmaceutical compositions and article of manufacture, according to the present invention, may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Protein tyrosine phosphatases (PTPs) are a large family of transmembrane or intracellular enzymes that dephosphorylate substrates involved in numerous signal transduction pathways (Fischer et al., Science 253:401-406, 1991; Anderson et al., Mol. Cell. Biol. 21: 7117-7134, 2001; Neel and Tonks, Curr. Opin. Cell. Biol. 9: 193-204, 1997; and Fischer, Adv. Enzyme. Regul. 39: 359-369, 1999).

The involvement of the intracellular protein tyrosine phosphatase-1B (PTP-1B) in regulating insulin and fat metabolism has been previously reported. Elchebly et al. (Science 283: 1544-1548, 1999) and Klaman et al. (Mol. Cell. Biol. 20: 5479-5489, 2000) reported that mice lacking the PTP-1B gene (PTP-1B knock out) developed increased insulin sensitivity and resistance to diet-induced obesity. The enhanced insulin sensitivity of the PTP-1B knock out mice was also evident in glucose and insulin tolerance tests.

The involvement of the receptor-type protein tyrosine phosphatase LAR in insulin regulation has also been reported. Hashimoto et al. (J. Biol. Chem. 267: 13811-13814, 1992) suggested that LAR might play a role in regulation of insulin receptors in intact cells. Ren et al. (Diabetes 47: 493-497, 1998) reported that LAR knock out mice exhibited profound defects in glucose homeostasis.

U.S. Pat. No. 6,410,556 teaches using novel compounds which inhibit PTP-1B, CD45, SHP-1, SHP-2, PTPα, LAR and HePTP and thus can be utilized for treating type I diabetes, type II diabetes, impaired glucose tolerance, insulin resistance, obesity, and other diseases.

U.S. Pat. No. 6,583,126 teaches novel compounds which inhibit PTP-1B and thus can be utilized for treating PTP-1B mediated diseases, including diabetes, obesity, and diabetes-related diseases.

Although members of the tyrosine phosphatase protein family have been linked to insulin regulation and obesity, the involvement of protein tyrosine phosphatase epsilon (PTPe) in body mass regulation has not been described or suggested in prior art.

While reducing the present invention to practice, the present inventors uncovered that modifying the activity or expression of PTPe can be used to modulate a body weight and/or fat content of a subject.

As is illustrated in Examples 1-4 of the Examples section which follows, the inventors of the present invention surprisingly uncovered that PTPe deficiency results in a substantial reduction of total body weight and of body fat content in laboratory animals. Specifically, downregulation of PTPe expression by gene knock-out, led to marked decrease of total body weight and fat content in male and female mice. In addition, such decrease of total body weight and fat content is observed in young mice (2-3 months old; Example 3), mature mice (10-11 months old; Example 1) and aged mice (14-15 months old; Example 2). Furthermore, downregulation of PTPe effectively suppresses increase in total body weight and fat content in estrogen-deficient female mice following ovariectomy, as illustrated in Examples 1-3. These observations clearly indicate that PTPe plays a major role in modulating fat metabolism and body weight changes in animals.

Thus, according to one aspect of the present invention, there is provided a method of modulating a body weight and/or fat content of a subject. The method is effected by modifying an activity or expression of PTPe in the subject so as to modulate the body weight of the subject.

The PTPe modified by the method of the present invention can be either a receptor type PTPe or a non-receptor type PTPe, examples of which include RPTPe, cyt-PTPe, p65 and p67.

As used herein the phrase “fat content” refers to the percentage of body weight attributed to fat tissue.

As used herein the phrase “modifying an activity or an expression” refers to partially or fully inhibiting or increasing activity or expression of PTPe.

Inhibiting PTPe activity or expression can be achieved by an agent such as an antibody or an antibody fragment capable of specifically binding PTPe. Preferably, the antibody specifically binds at least one epitope of a PTPe. As used herein, the term “epitope” refers to any antigenic determinant on an antigen to which the paratope of an antibody binds.

Epitopic determinants usually consist of chemically active surface groupings of molecules such as amino acids or carbohydrate side chains and usually have specific three dimensional structural characteristics, as well as specific charge characteristics.

The term “antibody” as used in this invention includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (1972)]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Alternately, or additionally, the agent capable of partially or completely inhibiting activity of PTPe may include an enzyme which cleaves PTPe thereby reducing its activity or rendering it inactive by for example relegating it to a subcellular organelle/location thus rendering incapable of exerting its biological effect. This enzyme may be, for example, calpain (Gil-Henn et al., Regulation of RPTP alpha and PTP epsilon by calpain-mediated proteolytic cleavage. J. Biol. Chem. 276, 31772-31779, 2001).

Another agent capable of downregulating a PTPe is a small interfering RNA (siRNA) molecule. RNA interference is a two step process. the first step, which is termed as the initiation step, input dsRNA is digested into 21-23 nucleotide (nt) small interfering RNAs (siRNA), probably by the action of Dicer, a member of the RNase III family of dsRNA-specific ribonucleases, which processes (cleaves) dsRNA (introduced directly or via a transgene or a virus) in an ATP-dependent manner. Successive cleavage events degrade the RNA to 19-21 bp duplexes (siRNA), each with 2-nucleotide 3′ overhangs [Hutvagner and Zamore Curr. Opin. Genetics and Development 12:225-232 (2002); and Bernstein Nature 409:363-366 (2001)].

In the effector step, the siRNA duplexes bind to a nuclease complex to from the RNA-induced silencing complex (RISC). An ATP-dependent unwinding of the siRNA duplex is required for activation of the RISC. The active RISC then targets the homologous transcript by base pairing interactions and cleaves the mRNA into 12 nucleotide fragments from the 3′ terminus of the siRNA [Hutvagner and Zamore Curr. Opin. Genetics and Development 12:225-232 (2002); Hammond et al. (2001) Nat. Rev. Gen. 2:110-119 (2001); and Sharp Genes. Dev. 15:485-90 (2001)]. Although the mechanism of cleavage is still to be elucidated, research indicates that each RISC contains a single siRNA and an RNase [Hutvagner and Zamore Curr. Opin. Genetics and Development 12:225-232 (2002)].

Because of the remarkable potency of RNAi, an amplification step within the RNAi pathway has been suggested. Amplification could occur by copying of the input dsRNAs which would generate more siRNAs, or by replication of the siRNAs formed. Alternatively or additionally, amplification could be effected by multiple turnover events of the RISC [Hammond et al. Nat. Rev. Gen. 2:110-119 (2001), Sharp Genes. Dev. 15:485-90 (2001); Hutvagner and Zamore Curr. Opin. Genetics and Development 12:225-232 (2002)]. For more information on RNAi see the following reviews Tuschl ChemBiochem. 2:239-245 (2001); Cullen Nat. Immunol. 3:597-599 (2002); and Brantl Biochem. Biophys. Act. 1575:15-25 (2002).

Synthesis of RNAi molecules suitable for use with the present invention can be effected as follows. First, the PTPe mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www.ambion.com/techlib/tn/91/912.html).

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene. siRNA sequences which can be used to downregulate PTPe expression according to the teaching of the present invention are set forth in SEQ ID NOs: 1-3 and 7-10.

As is shown in Example 5 of the Examples section below, an siRNA molecule generated according to the teachings of the present invention was highly effective in downregulating expression of PTPe. In addition, this Example also demonstrates the high selectivity of this siRNA in that it is capable of inhibiting expression of the two major protein isoforms of PTPe—the receptor-type and the non-receptor type forms (RPTPe and cyt-PTPe, respectively) and yet has no inhibitory effect on the highly-related RPTP alpha which lacks the target sequence.

Another agent capable of downregulating a PTPe is a DNAzyme molecule capable of specifically cleaving an mRNA transcript or DNA sequence of the PTPe. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al, 20002, Abstract 409, Ann Meeting Am Soc Gen Ther www.asgt.org). In another application, DNAzymes complementary to bcr-abl oncogenes were successful in inhibiting the oncogenes expression in leukemia cells, and lessening relapse rates in autologous bone marrow transplant in cases of CML and ALL.

Inhibition of PTPe expression can also be effected by using an antisense polynucleotide capable of specifically hybridizing with an mRNA transcript encoding the PTPe thereby specifically inhibiting translation of the PTPe transcripts.

Design of antisense molecules which can be used to efficiently inhibit PTPe expression must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft, J Mol Med 76: 75-6, 1998; Kronenwett et al., Blood 91: 852-62, 1998; Rajur et al., Bioconjug Chem 8: 935-40, 1997; Lavigne et al., Biochem Biophys Res Commun 237: 566-71, 1997; and Aoki et al., Biochem Biophys Res Commun 231: 540-5), 1997].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9, 1999].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al., Nature Biotechnology 16, 1374-1375, 1998).

Several clinical trials have demonstrated safety, feasibility and activity of antisense oligonucleotides. For example, antisense oligonucleotides suitable for the treatment of cancer have been successfully used (Holmund et al., Curr Opin Mol Ther 1:372-85, 1999), while treatment of hematological malignancies via antisense oligonucleotides targeting c-myb gene, p53 and Bcl-2 had entered clinical trials and had been shown to be tolerated by patients (Gerwitz Curr Opin Mol Ther 1:297-306, 1999).

More recently, antisense-mediated suppression of human heparanase gene expression has been reported to inhibit pleural dissemination of human cancer cells in a mouse model (Uno et al., Cancer Res 61:7855-60, 2001).

Thus, the current consensus is that recent developments in the field of antisense technology which, as described above, have led to the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation. SEQ ID NO: 4 exemplifies one antisense nucleotide sequence which can be utilized to downregulate expression of PTPe.

The antisense sequences may include a ribozyme sequence which is capable of cleaving transcripts encoding PTPe, thereby preventing translational of those transcripts into functional PTPe. Such a ribozyme is readily synthesizable using solid phase oligonucleotide synthesis.

Ribozymes are being increasingly used for the sequence-specific inhibition of gene expression by the cleavage of mRNAs encoding proteins of interest [Welch et al., “Expression of ribozymes in gene transfer systems to modulate target RNA levels.” Curr Opin Biotechnol. 1998 October; 9(5):486-96]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., “Ribozyme gene therapy for hepatitis C virus infection.” Clin Diagn Virol. 1998 Jul. 15; 10(2-3):163-71.]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-R (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

Alternately or additionally, the agent may include a non-functional analogue of at least a catalytic or binding portion of the PTPe. This non-functional analogue may be capable of, for example, binding a site of cyt-PTPe activity, such as Src.

As mentioned hereinabove, the present invention also envisages upregulation of PTPe activity or expression, since such upregulation is expected to increase total body weight and fat content, a feature which finds utility when increasing body weight and/or fat content is desired.

Upregulation of PTPe expression levels can be effected by delivering to a subject an exogenous polynucleotide sequence designed and constructed to express at least a functional portion of the PTPe in the subject.

In order to generate a polynucleotide construct capable of expressing at least a functional portion of PTPe according to the present invention, a polynucleotide segment encoding PTPe or a functional portion thereof can be ligated into a commercially available expression vector system suitable for transforming mammalian cells and for directing the expression of PTPe within the transformed cells. Preferably the construct will further include a suitable promoter.

It will be appreciated that such commercially available vector systems can easily be modified via commonly used recombinant techniques in order to replace, duplicate or mutate existing promoter or enhancer sequences and/or introduce any additional polynucleotide sequences such as for example, sequences encoding additional selection markers or sequences encoding reporter polypeptides.

Suitable mammalian expression vectors for use with the present invention include, but are not limited to, pcDNA3, pcDNA3.1(+/−), pZeoSV2(+/−), pSecTag2, pDisplay, pEF/myc/cyto, pCMV/myc/cyto, pCR3.1, which are available from Invitrogen, pCI which is available from Promega, pBK-RSV and pBK-CMV which are available from Stratagene, pTRES which is available from Clontech, and their derivatives.

Increasing PTPe expression levels can also be effected by an agent(s) which increases expression of endogenous PTPe. For Example, cyt-PTPe expression levels can be increased via serum after serum starvation, TPA, and basic FGF which have been shown to be effective in increasing PTPe expression in fibroblast cultures [Elson A. and Leder, P. Identification of a cytoplasmic, phorbol ester-inducible isoform of the protein tyrosine phosphatase Epsilon. Proc. Natl. Acad. Sci. USA 92: 12235-12239,1995].

Alternatively, increasing PTPe activity may be promoted, for example, by a non-functional (i.e., non-catalytic) fragment of PTPe which is capable of binding and activating the enzyme.

Preferably, the agents described herein are administered to the subject via, for example, systemic administration routes or via oral, rectal, transmucosal (especially transnasal), intestinal or parenteral administration routes. Systemic administration includes intramuscular, subcutaneous and intramedullary injections as well as intrathecal, direct intraventricular, intravenous, inrtaperitoneal, intranasal, or intraocular injections.

As is illustrated in the Examples section which follows, inhibiting the activity or expression of PTPe decreases the body weight and/or fat content of the subject, while increasing activity or expression of PTPe is expected to increase body weight and/or fat content of the subject.

Thus, the present methodology of modifying an activity or an expression of PTPe can also be used to treat or prevent weight disorders.

As used herein the phrase “weight disorder” refers to a condition which is associated with an abnormal body weight, including disorders associated with overweight such as obesity, or disorders associated with underweight such as anorexia or cachexia.

As used herein the phrase “treat or prevent” refers to alleviating, inhibiting, prohibiting, restraining, or reversing progression of a disease or disorder. Accordingly, treating an overweight disorder includes inhibiting weight gain or inducing weight loss of a subject, while treating an underweight disorder includes inhibiting weight loss or inducing weight gain of a subject.

A weight disorder may considerably increase the risk of developing, or enhance progression of a number of diseases, which are referred to herein as “weight disorder related diseases”.

Thus, apart for being suitable for treating weight disorders, the present invention can also be utilized to treat related diseases which oftentimes directly result from the weight disorders.

For example, an excess body weight greater than 30% doubles the incidence of coronary diseases in subjects less than 50 years old; an excess body weight of 20% doubles the risk of high blood pressure; an excess body weight of 30% triples the risk of developing non-insulin dependent diabetes; and an excess body weight of 30% multiplies the risk of developing hyperlipidemias by six fold. Accordingly, weight disorder related diseases include, but not limited to, atherosclerosis, hypertension, stroke, myocardial infraction, Type II or non-insulin dependent diabetes mellitus, pancreatitis, hypercholestrosaemia, hypertriglyceridaema and hyperlipidemia.

The above described agents, whether selected for positive or negative regulation of PTPe activity or expression, can be administered to the subject per se or as part (active ingredient) of a pharmaceutical composition.

As used herein a “pharmaceutical composition” refers to a preparation of one or more of the active ingredients or agents described herein with other chemical components such as physiologically suitable carriers and excipients. The purpose of a pharmaceutical composition is to facilitate administration of a compound to an organism.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, inrtaperitoneal, intranasal, or intraocular injections.

Although not presently preferred, one may administer the pharmaceutical composition in a local rather than systemic manner, for example, via injection of the pharmaceutical composition directly into a tissue region of a patient. Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (e.g. antisense oligonucleotide) effective to prevent, alleviate or ameliorate symptoms of a disorder (e.g., excessive overweight or excessive underweight) of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. Preferably, a dose is formulated in an animal model to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to levels of the active ingredient are sufficient to substantially affect the body weight or fat content of an individual. Dosages necessary to achieve the desired effect will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

Pharmaceutical compositions according to the present invention may further include other anti obesity compounds, such as, but not limited to appetite suppressants, fenfluramine, dexfenfluramine, phentiramine, sulbitramine, orlistat, pyrazolecarboxamide, neuropeptide Y5 receptor antagonists, leptin, β-3-adrenergic receptor agonists, serotonin agonists and PPARγ antagonists, to act synergistically or additively with an agent as described hereinabove, thereby increasing the overall efficacy of the pharmaceutical composition.

Alternatively, pharmaceutical compositions according to the present invention may further include underweight control substances such as nutritional supplements, appetizer stimulants and/or weight promoters such as, but not limited to norepinephrine agonists, neuropeptide Y, melanocyte concentrating hormone, modafinil, insulin and amylin to act synergistically or additively with an agent as described hereinabove, thereby increasing the overall efficacy of the pharmaceutical composition.

Since the present invention demonstrates a correlation between the level of PTPe activity or expression and the body weight or fat content of an individual the present invention further provided a method of identifying a drug candidate suitable for modulating body weight and/or fat content of a subject.

Identification of a drug candidate, according to the present invention may be accomplished by screening a plurality of molecules for a molecule, which is capable of modifying an activity or expression of PTPe as described hereinabove. Such screening identifies molecules capable of modifying the activity or expression of PTPe with each of the identified molecules becoming a drug candidate. Some of these candidates will eventually become agents capable of modifying an activity or an expression of PTPe as described hereinabove, either alone, or as an active ingredient in a pharmaceutical composition.

The following section describes in detail one configuration of a screening procedure which can be used for identifying a peptide or a polypeptide drug candidate suitable for modulating body weight and/or fat content of a subject and for treatment or prevention of a weight disorder and related diseases.

A peptide or polypeptide sequence derived from cyt-PTPe is immobilized on a suitable substrate (e.g. agarose or sepharose beads). The peptide may be synthesized or isolated from natural sources according to established protocols. A complex mixture of peptides, for example a crude proteolysed cell extract, is incubated with the substrate beads bearing the target molecule. The substrate beads bearing the target molecule are then washed to remove molecules which bind with low affinity. High affinity binding molecules are then eluted and collected. These high affinity binding molecules are then re-incubated with substrate beads to which an irrelevant target molecule (e.g. a beta globin derived peptide) has been bound. It will be appreciated that the stringency of the screening may be regulated to a great degree by choice of the irrelevant target molecule used in the re-incubation. For example, use of the receptor form of PTPe (RPTPe) instead of a beta globin derived peptide will greatly increase the stringency of the assay and the specificity of the purified peptide to the cytosolic form of PTPe. The supernatant, containing molecules which did not bind during this second incubation, is collected and purified.

As an example, purification might include gel filtration chromatography and/or ion exchange chromatography and SDS-PAGE of eluted fractions followed by blotting to a membrane (e.g. PVDF or nitrocellulose), incubation with the peptide homologous to a cyt-PTPe binding site employed in the first step of the screening and immunodetection employing a primary antibody with specificity to the same peptide.

Duplicate blots would be subjected to similar treatment using an irrelevant target molecule (e.g. a keyhole limpet hemocyanin derived peptide) instead of the peptide homologous to a cyt-PTPe binding site and a primary antibody against keyhole limpet hemocyanin

Molecules which gave a positive result on the first blot and a negative result on the second blot would become candidates for additional purification steps, for example, HPLC purification of peptides eluted from PAGE gels. Once purified to homogeneity, these peptides can be sequenced and either produced synthetically, produced using recombinant DNA technology, or derived from natural sources (via, for example, proteolysis).

The present invention provides a novel approach for modulating body weight and/or fat content of a subject and for treatment or prevention of a weight disorder and related diseases. The methods of the present invention rely upon modifying an activity or an expression of a PTPe. Further, the present invention provides pharmaceutical compositions, articles of manufacture useful in practice of the methods and methods of identifying suitable drug candidates.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W.H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984); “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996) and Parfitt et al. (1987). Bone histomorphometry: standardization of nomenclature, symbols, and units. Report of the ASBMR Histomorphometry Nomenclature Committee. J Bone Miner Res 2 (6), 595-610; all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Methods

Animals: Gene-targeted mice lacking PTPe have been previously described (Peretz et al., EMBO J. 19: 4036-4045, 2000). Wild type and knock-out mice were of the same mixed C57B1/6×129 genetic background. All handling and manipulation of mice was done in accordance with applicable laws and accepted guidelines for animal welfare.

Maintenance: Mice were housed in rooms equipped with HEPA-filtered air at 16-26° C. with a relative humidity of 30-70%. Illumination was approximately 300 lumens/m² at 1 m above floor level on a 12-hour light/dark cycle. Mice were housed three or four per cage. Cages were clear polycarbonate plastic, 29×19×13 cm dimensions. The bedding material was irradiated corncob bedding (Bed-O-Cob, The Anderson's Maumee, Ohio, USA). Mice were given ad libitum access to deionized water and to regular rodent diet (Certified Picolab Rodent 20 Lab Diet, PMI Nutrition International, Brentwood, Mo.).

Surgery: The hair on the dorsal abdominal surface was shaved and incision made in both sides of the lateral abdominal region. Incisions were made through the abdominal musculature and the ovaries were either removed (ovariectomy) or manipulated (Sham). The skin incisions were closed using surgical staples.

Statistical analysis: Statistical significance was determined by unpaired one-tailed t-test.

### Example 1

### Effect of PTPe and Estrogen Deficiencies on Body Weight and Fat Content of 10-11 Month Old Female Mice

Mature (10-11 months old) female mice were treated as follows: (i) wild type (WT)—Sham operated (Sham; 8 animals); (ii) WT—ovariectomy (OVX; 8 animals); (iii) PTPe knock out (KO)—Sham (7 animals); and KO-OVX (8 animals). The animals' weight and percent body fat were determined before surgery (OVX or Sham) and following surgery for eight weeks. The percent body fat was determined by DEXA scanning using PIXImus instrument software (Lunar Corp., Madison, Wis.).

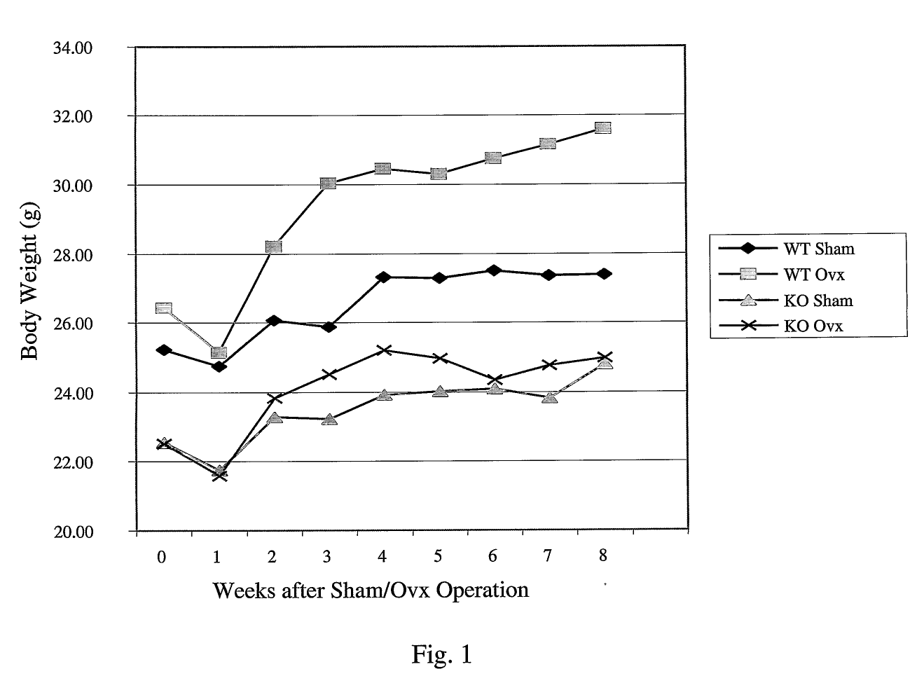

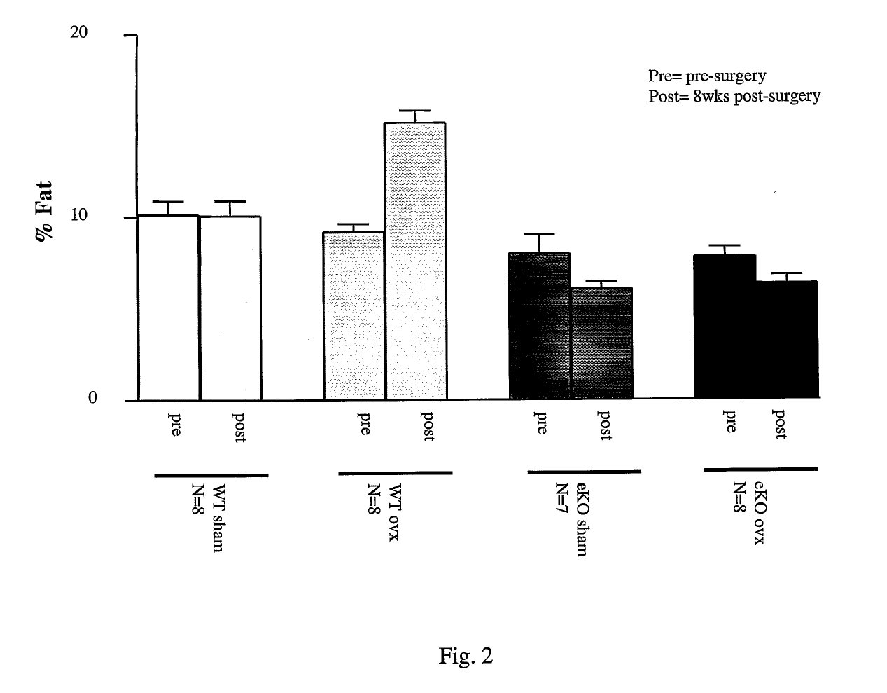

FIGS. 1-2 illustrate the effect of PTPe and estrogen deficiencies on the body weight and the body fat content of 10-11 months old female mice. FIG. 1 shows that wild type mice had significantly (p<0.05) higher body weights, as compared with PTPe knock-out mice, both prior to surgery and during the 8-week period following surgery. Furthermore, ovariectomy of wild type mice induced a substantial (25.8%) and statistically significant (p<0.05) increase in body weight, for 8 weeks following surgery, while its effect on PTPe knock-out mice following ovariectomy was much smaller (15.8% increase in body weight) and non-significant. In addition, the weight difference between ovariectomized and Sham-operated wild type mice increased constantly during the 8 week period after surgery. On the other hand, the weight difference between ovariectomized and Sham-operated PTPe knock-out mice became indistinguishable five weeks after surgery. Apparently, the PTPe knock-out mice became resistant to the ovariectomy-induced weight gain.

The changes in the body fat content following surgery are illustrated in FIG. 2 and are further summarized in Table 1 hereinbelow. As shown in FIG. 2, prior to surgery the body fat content (percent) of wild type mice was significantly higher (p<0.05) than that of PTPe knock-out mice. Table 1 shows that wild type mice which underwent Sham surgery did not gain body fat during the eight-week period post surgery, while the body fat content of the respective ovariectomized wild type mice increased by 66.1% (p<0.05). On the other hand, ovariectomy did not have any effect on the body fat content of the PTPe knock-out mice. These results indicate that PTPe plays a role in modulating body weight and fat metabolism of mature estrogen-deficient female mice.

TABLE 1
Changes in body fat content (%) of female mice
before and after¹ ovariectomy or sham surgery

| | | Change in body | |
|---|---|---|---|
| Genotype | Treatment | fat content (%) | |
| Wild Type | Sham | −0.45 | A² |
| Wild Type | Ovariectomy | 66.11 | B |
| PTPe knock-out | Sham | −17.91 | A |
| PTPe knock-out | Ovariectomy | −17.75 | A |

¹Eight weeks after ovariectomy or sham operation.
²Values followed by different letters were significantly different (p < 0.05).

### Example 2

### Effect of PTPe Deficiency on Body Weight and Fat Content of 14-15 Months Old Female Mice

Aged (14-15 months old) wild type and knock-out female mice were weighed and analyzed for the percent body fat, as described above.

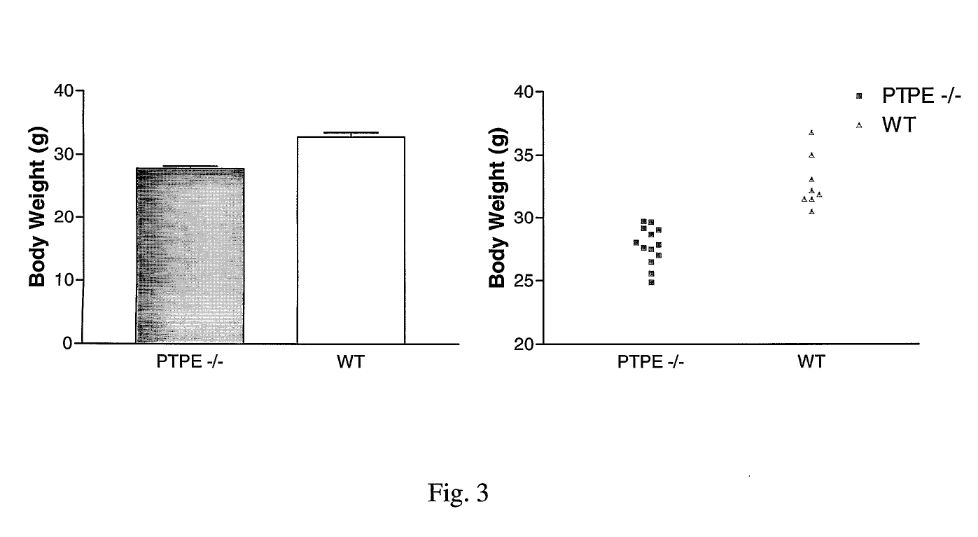

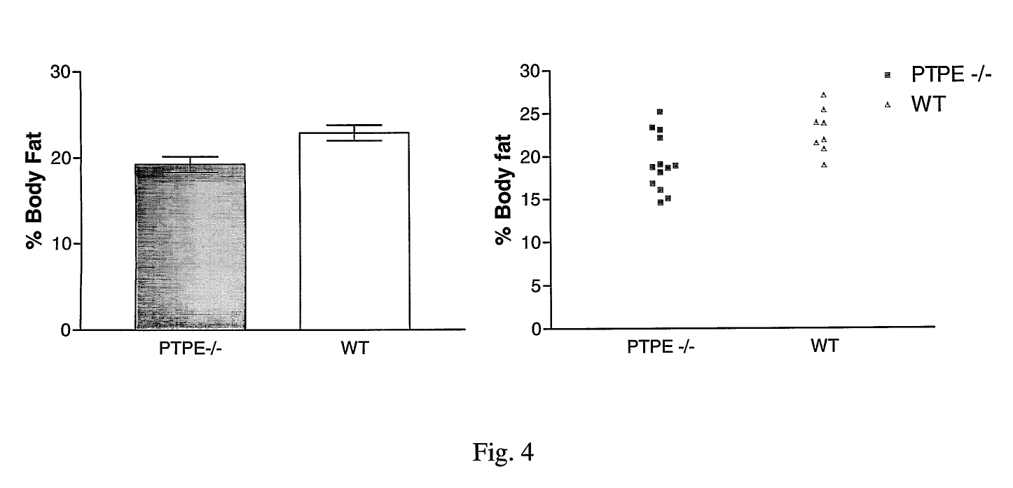

FIGS. 3-4 illustrate the effect of PTPe deficiency on the body weight and body fat content of female mice. These Figures show that PTPe knock out mice averaged 15.4% lower body weight than wild type mice (FIG. 3; p<0.05) and 16.0% lower body fat content than the wild type mice (FIG. 4; p<0.05).

These results indicate that PTPe plays a role in modulating body weight and fat metabolism of aged female mice and that this effect is independent of the effect of estrogen.

### Example 3

### Effect of PTPe and Estrogen Deficiencies on Body Weight and Fat Content of 2-3 Months Old Female Mice

Young (2-3 months old) female mice were treated as follows: WT-Sham, WT-OVX, KO-Sham and KO-OVX. Five animals (replications) were used for each treatment. The body weight and food-intake rate values were determined weekly for 10 weeks following surgery. At the end of the 10 week follow-up period all mice were sacrificed and their abdominal fat pads were removed and weighed.

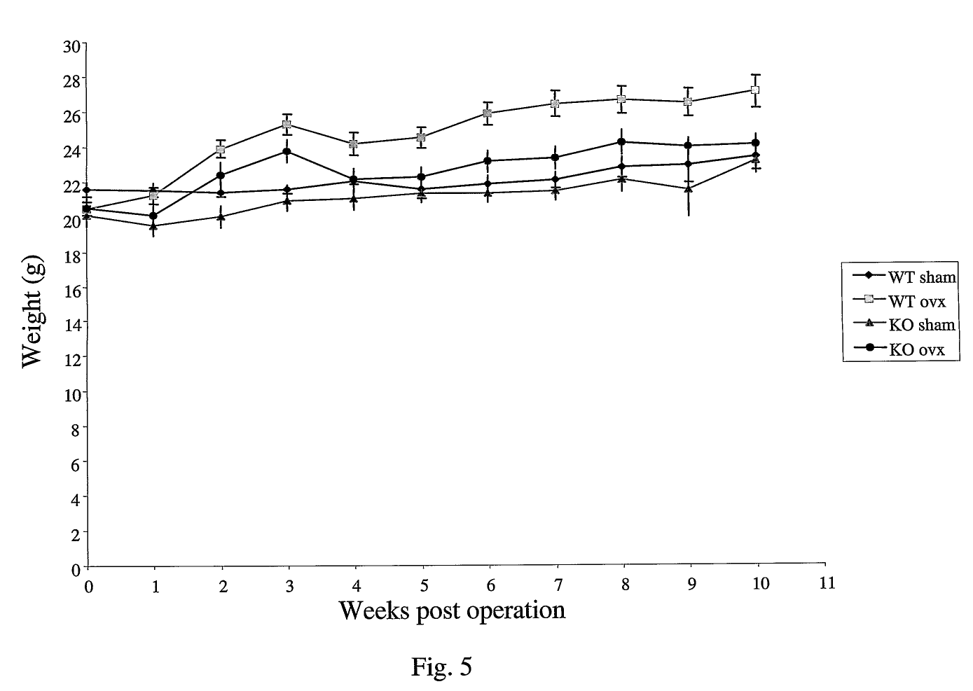

FIG. 5 illustrates the effects of PTPe and estrogen deficiencies on the body weight of 2-3 months old female mice. This Figure shows that ovariectomy caused a significant increase (p<0.05) in the body weight of wild type mice during the 2-10 week period post surgery (15.9% increase by week 10). On the other hand, ovariectomy of PTPe knock-out mice did not significantly affect the body weight of PTPe knock-out mice during the same period.

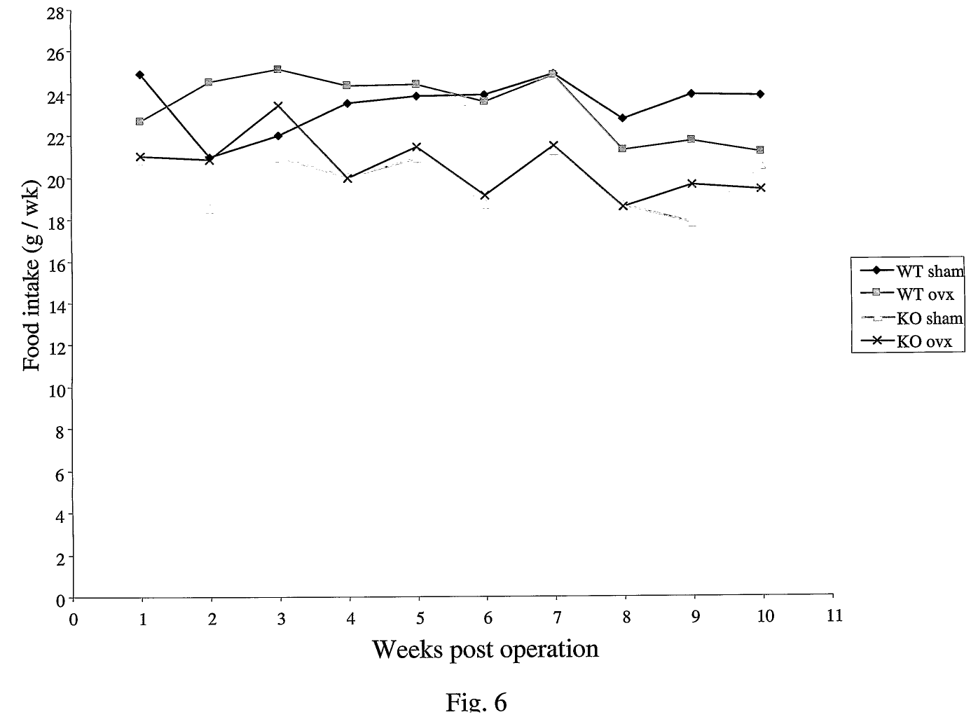

FIG. 6 illustrates the effects of PTPe and estrogen deficiencies on the rate of food intake by 2-3 months old female mice. This Figure shows that ovariectomized PTPe knock-out mice consumed less food than ovariectomized wild-type mice (p<0.05) during week 2, and 4-9 post surgery (p<0.05). Similarly, Sham-operated PTPe knock-out mice consumed less food than Sham-operated wild-type mice during week 2, and 4-9 post surgery (p<0.05). Overall, the PTPe knock-out mice consumed substantially less food than the wild type mice, irrespective of the ovariectomy operation.

Tables 2-3 hereinbelow show that the fat pad weight of ovariectomized wild type mice, determined 10 weeks post surgery, was substantially higher (increase of 214.4%; p=0.0006) then the fat pad weight of the respective Sham-operated wild type mice. On the other hand, ovariectomy of PTPe knock-out mice resulted in a much smaller increase of fat pad weight 10 weeks post surgery (increase of 36.5%; p=0.07-0.09).

These results indicate that PTPe plays a key role in modulating body weight and fat metabolism of young estrogen-deficient female mice.

TABLE 2
The effects of PTPe deficiency and ovariectomy
on the abdominal fat weight of female mice

Fat Pad Weight

| Genotype | Treatment³ | Mean (g) | Standard Error |
|---|---|---|---|
| KO¹ | Sham⁴ | 0.293 | 0.048 |
| KO | OVX⁵ | 0.4 | 0.019 |
| WT² | Sham | 0.222 | 0.0386 |
| WT | OVX | 0.698 | 0.079 |

¹KO = PTPe knock out.
²WT = wild type.
³Treatment was performed on 2-3 months old female mice.
⁴Sham = ovaries manipulation only.
⁵OVX = ovariectomy.

TABLE 3
Comparisons between treatment groups

| Comparison | Difference (%) | p value |
|---|---|---|
| KO-sham vs. KO-OVX | 36.5 | 0.07-0.09 |
| KO-sham vs. WT-sham | 24.0 | NS¹ |
| KO-OVX vs. WT-OVX | 74.5 | 0.0024**² |
| WT-sham vs. WT-OVX | 214.4 | 0.0006** |

¹NS = not significant.
²**= highly significant (p < 0.01).

Overall, the observed weight gain in wild type mice following ovariectomy is typical and consistent with the condition commonly observed in rodents undergoing a similar procedure. This weight gain is attributed to an excessive accumulation of fatty tissue, which is supported by substantial increases in the percentage of total body fat and the fat pad weight of ovariectomized wild type mice. However, the lack of such increases in ovariectomized PTPe-deficient mice was surprising and unexpected. These results clearly indicate that PTPe plays a major role in modulating estrogen-sensitive fat tissue metabolism. Furthermore, the lower body weight and the lower body fat content of untreated, or Sham-operated PTPe-deficient mice indicate that PTPe is capable of modulating general fat metabolism independently of estrogen.

### Example 4

### PTPe Deficiency Decreases Body Weight of Male and Female Mice

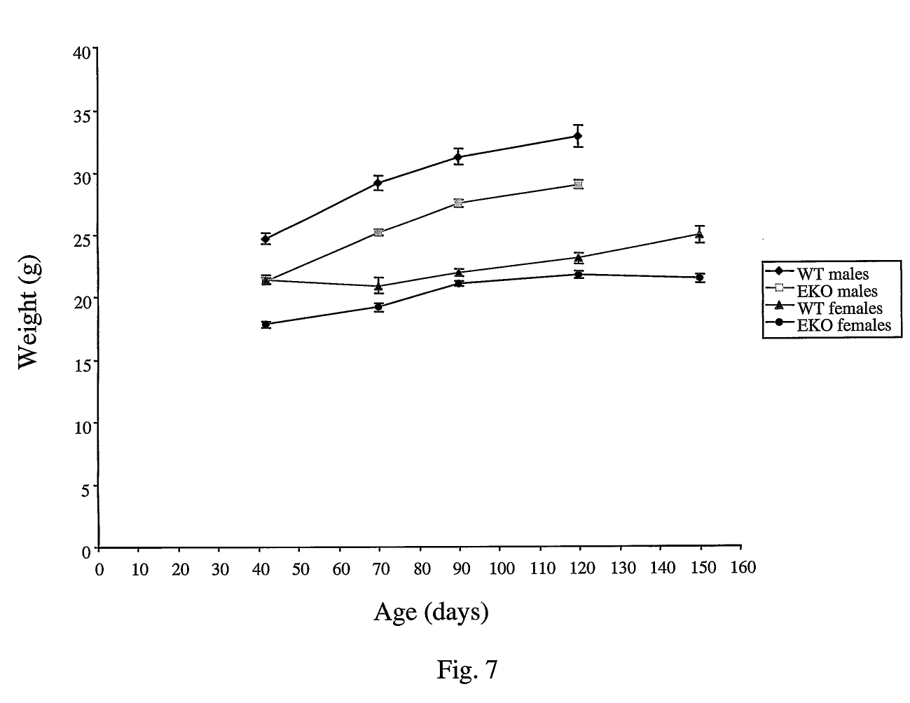

In order to determine if the PTPe-induced effect on body weight is limited to females only, the body weights of male WT, male PTPe knock-out, female WT and female PTPe knock-out were comparatively evaluated during a 108 days period. The results, presented in FIG. 7, show that PTPe-deficient mice of both sexes were slightly (4-15%), but significantly (p<0.05) smaller than their respective wild types throughout the trial period.

These results indicate that the effect on modulating total body weight and fat metabolism in mice is not sex limited.

Overall, the results presented in the Examples hereinabove uncover that PTPe is a powerful modulator of fat metabolism and subsequent body weight in male or female animals, as well as in young or old animals. Thus, modifying an activity or expression of PTPe can be an effective method of treating or preventing a variety of weight disorders and related diseases in a subject of need.

### Example 5

### siRNA Inhibition of PTPe Expression

SiRNA-mediated inhibition of PTPe expression was established by selecting several PTP DNA sequences according to criteria well-established in the art. Specifically, the sequences selected were 19 base pairs long, within the coding region, and have a GC content selected from a range of 30-70% with all four bases similarly represented. The selected (core) sequences were then each flanked by an AA dinucleotide at the 5′ end and a TT dinucleotide at the 3′ end generating an oligomer of 23 base pairs.

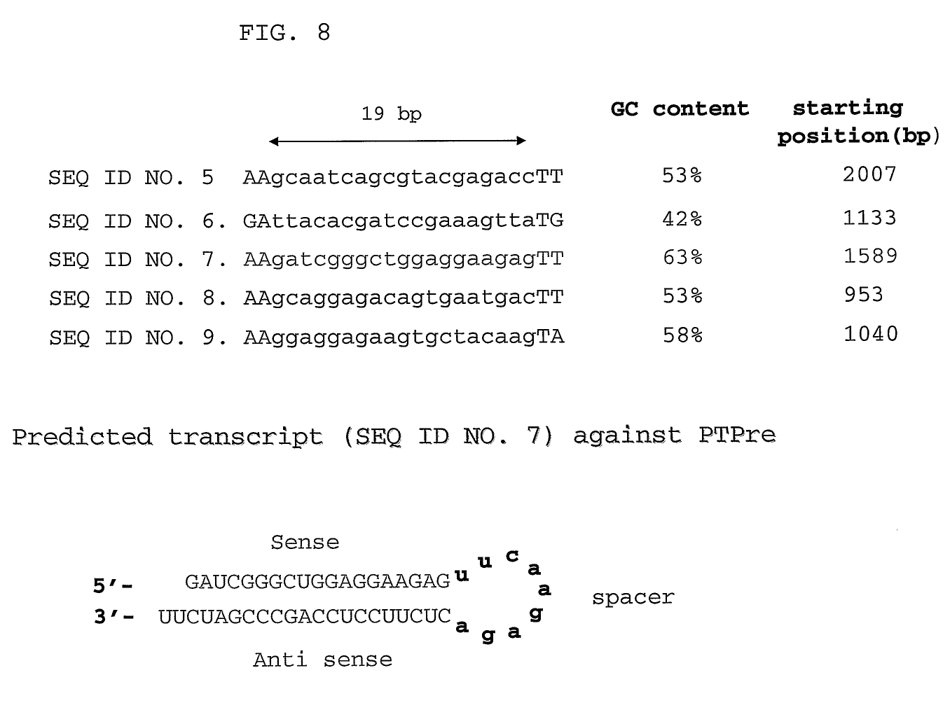

For in cell transcription of the siRNA, a longer oligomer is synthesized. A random sequence of 9 base pairs (typically the sequence ttcaagaga, SEQ ID NO. 10) is used to link between the selected siRNA sequence (flanked with dinucleotides) and an inverted repeat thereof. The final molecule (shown in FIG. 8, bottom) thus includes [sequence (23 bases)ttcaagaga (inverted repeat, 23 bases)].

Transcription of this molecule in cells results in an RNA molecule that folds over into a hairpin duplex, which is then further processed by Dicer and other endogenous enzymes to yield the active RNA species.

To enable in cell transcription, the selected (and flanked) sequence and its reverse compliment are synthesized, denatured, annealed, and cloned into the pSUPER vector at sites flanking a random loop forming sequence (such as SEQ ID NO:10). The resulting pSUPER-based vector is transfected into cells and the expression level of the targeted gene product is assayed at the RNA or DNA levels.

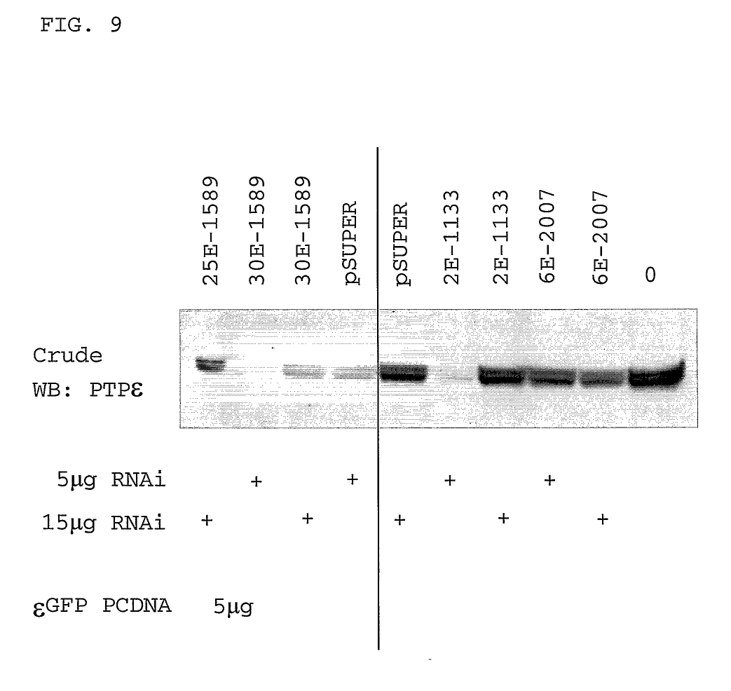

Several candidate sequences were selected for in-situ experiments (SEQ ID NOs: 5-7) according to a computerized analysis (http://sinc.sunysb.edu/Stu/shilin/mai.html). As seen in FIG. 9, co-transfection of 5 micrograms of the pSUPER-based plasmid containing SEQ ID NO:7 (designated as 1589) significantly inhibited PTPe expression at the protein level relative to cells transfected with empty pSUPER plasmid alone or not transfected with any pSUPER-based derivative. Greater amounts of the pSUPER-based siRNA (15 micrograms) did not inhibit PTPe expression well, a phenomenon often observed with siRNA inhibition. siRNA molecules 1133 (SEQ ID NO:6) also exhibited some downregulation while molecule 2007 (SEQ ID NO:5) produced no detectable downregulation of PTPe expression.

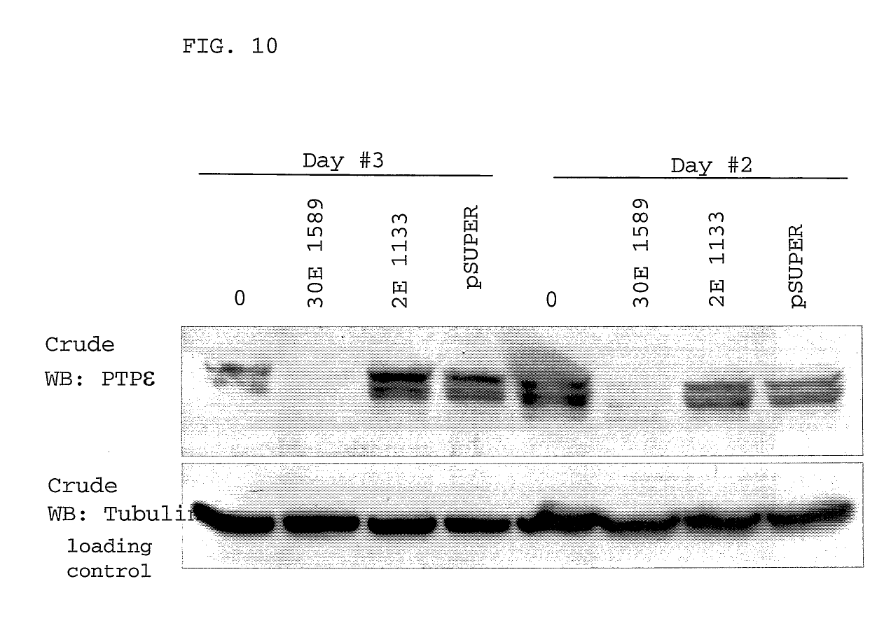

FIG. 10 shows that similar results were obtained when PTPe was expressed in cells without fused GFP protein; this Figure also indicates that analysis 2 or 3 days following transfection (the 3 day period previously being the standard wait) produces similar results to those shown in FIG. 9.

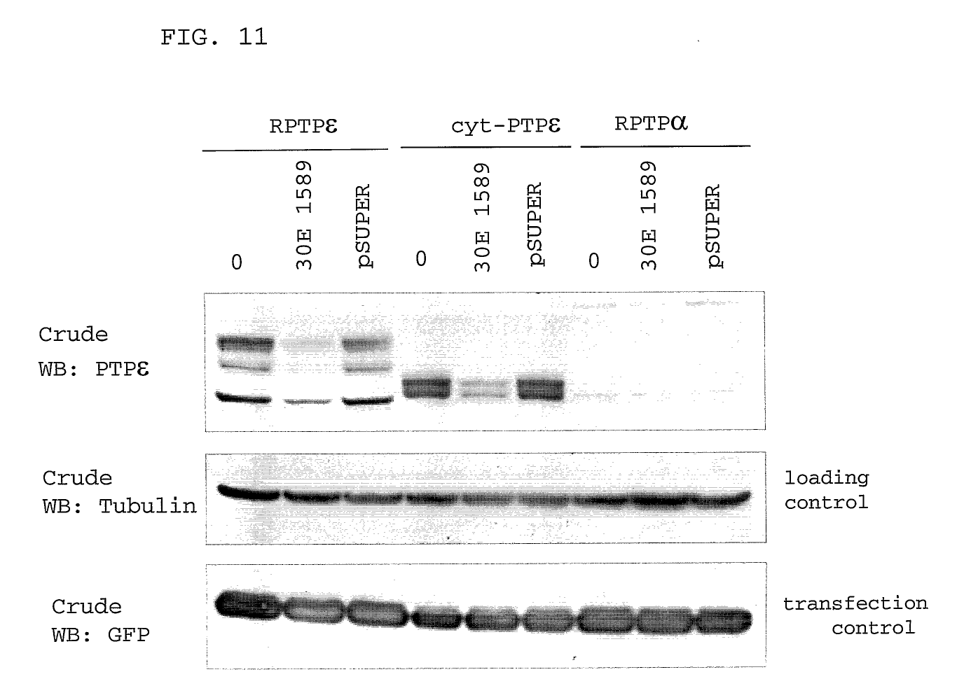

FIG. 11 shows that the effect of a SEQ ID NO:7-based siRNA molecule is specific to PTPe. Expression of this molecule in 293 cell inhibits expression of the two major protein isoforms of PTPe—the receptor-type and the non-receptor type forms (RPTPe and cyt-PTPe, respectively). This is expected since both forms share the sequence corresponding to SEQ ID NO:7. In contrast, no inhibitory effect is observed for the highly-related RPTP alpha (FIG. 11) which lacks the siRNA-derived sequence.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent application or sequence identified by their accession number was specifically and individually indicated to be incorporated herein by reference. In addition, citation or identification of any reference in this application shall not be construed as an admission that such reference is available as prior art to the present invention.

What is claimed is:

1. A method of modulating a body weight and/or fat content of a subject, comprising modifying an activity or expression of a protein tyrosine phosphatase epsilon (PTPe) thereby modulating the body weight and/or fat content of the subject.

2. (canceled)

3. The method of claim 1, wherein said PTPe is selected from the group consisting of RPTPe, cyt-PTPe, p65 and p67.

4. The method of claim 1, wherein said modifying includes at least partially inhibiting an activity or expression of said PTPe.

5. (canceled)

6. The method of claim 4, wherein said at least partially inhibiting said activity or expression of said PTPe is effected by introducing into the subject a small interfering RNA (siRNA) molecule.

7-8. (canceled)

9. The method of claim 1, wherein the subject is a human.

10. The method of claim 1, wherein said modifying includes increasing said activity or expression of said PTPe.

11-30. (canceled)

31. A method of treating or preventing a weight disorder in a subject, comprising modifying an activity or expression of PTPe, thereby treating or preventing the weight disorder in the subject.

32. The method of claim 31, wherein said weight disorder is selected from the group consisting of obesity, anorexia and cachexia.

33. (canceled)

34. The method of claim 31, wherein said PTPe is selected from the group consisting of RPTPe, cyt-PTPe, p65 and p67.

35. The method of claim 31, wherein said modifying includes at least partially inhibiting an activity or expression of said PTPe.

36. (canceled)

37. The method of claim 35, wherein said at least partially inhibiting said activity or expression of said PTPe is effected by introducing into the subject a small interfering RNA (siRNA) molecule.

38-39. (canceled)

40. The method of claim 31, wherein the subject is a human.

41. The method of claim 31, wherein said modifying includes increasing said activity or expression of said PTPe.

42-52. (canceled)

53. A method of treating or preventing a weight disorder related disease in a subject, comprising modifying an activity or expression of a PTPe thereby treating or preventing the weight disorder related disease in the subject.

54. The method of claim 53, wherein said weight disorder related disease is selected from the group consisting of atherosclerosis, hypertension, Type II or non-insulin dependent diabetes mellitus, pancreatitis, hypercholestrosaemia, hypertriglyceridaema and hyperlipidemia.

55. (canceled)

56. The method of claim 53, wherein said PTPe is selected from the group consisting of RPTPe, cyt-PTPe, p65 and p67.

57. The method of claim 53, wherein said modifying includes at least partially inhibiting an activity or expression of said PTPe.

58. (canceled)

59. The method of claim 57, wherein said at least partially inhibiting said activity or expression of said PTPe is effected by introducing into the subject a small interfering RNA (siRNA) molecule.

60-61. (canceled)

62. The method of claim 53, wherein said subject is a human.

63. The method of claim 53, wherein said modifying includes increasing said activity or expression of said PTPe.

64-80. (canceled)

81. The method of claim 6, wherein said small interfering RNA (siRNA) molecule comprises SEQ ID NO:7.

82. The method of claim 37, wherein said small interfering RNA (siRNA) molecule comprises SEQ ID NO:7.

83. The method of claim 59, wherein said small interfering RNA (siRNA) molecule comprises SEQ ID NO: 7.
